(19) United States
(12) Patent Application Publication
(10) Pub. No.: US 2023/0295082 A1
(43) Pub. Date: Sep. 21, 2023

(54) MODIFIED EZETIMIBE DRUG FOR CANCER TREATMENT

(71) Applicant: University of South Africa, Pretoria (ZA)

(72) Inventors: Monde Ntwasa, Johannesburg (ZA); Charmy Starnod Twala, Thabazimbi (ZA)

(21) Appl. No.: 18/019,067

(22) PCT Filed: Aug. 2, 2021
(86) PCT No.: PCT/IB2021/057046

§ 371 Date: Jan. 31, 2023

(30) Foreign Application Priority Data
Aug. 4, 2020 (ZA) 2020/04811

(51) Int. Cl. C07D 205/08

(52) U.S. Cl. CPC C07D 205/08

(57) ABSTRACT

The invention discloses a novel compound of Formula (I) or a pharmaceutically acceptable salt thereof that binds tightly to a hydrophobic binding pocket of MM, preventing binding of Mdm2 to the tumour suppressor p53 and increasing p53 levels. It further discloses the use of the compound or a pharmaceutically acceptable salt thereof to treat Mdm2 cancers and its use in the manufacture of a medicament.

### FIELD OF THE INVENTION

THIS INVENTION relates to an anti-cancer drug that is a modification of the drug ezetimibe, the modified drug having improved drug-like properties and being less vulnerable to metabolic enzymes than ezetimibe.

### BACKGROUND TO THE INVENTION

The tumour suppressor protein p53 is involved in a number of important biological process that are crucial for carcinogenesis, including the cell cycle, apoptosis, DNA repair, angiogenesis, glucose metabolism and innate immunity. It is a transcription factor that acts as a regulator of the oncogene Mouse Double Minute (Mdm2) in an auto regulatory feed-back loop. p53 activates expression of the Mdm2 gene and Mdm2 regulates p53 by controlling its transport out of the nucleus thereby making it unavailable to its gene targets. This inhibits its transcription function or promotes its degradation by proteasomes using ubiquitin-ligase activity.

Mdm2 has a hydrophobic binding pocket to which p53 binds by means of a peptide in its transactivation domain. This pocket is a key target for drugs that inhibit the p53-Mdm2 interaction. Small molecular drug design has attempted to formulate small drug-like molecules that can competitively target the Mdm2 p53-binding domain, disrupting the formation of Mdm2-p53 complexes and thus increasing the levels of reactive p53 in cancer cells to promote a p53-dependent cell death. These drug design studies have resulted in the synthesis of nutlins (nutlin-2, nutlin-3a and MI-219), of which nutlin-3a is the most potent with a very low toxicity profile (IC₅₀ 0.09 μM).

The applicant has previously shown that ezetimibe can bind more strongly than nutlins to the Mdm2 hydrophobic pocket. Furthermore, the applicant has shown that ezetimibe is toxic to cancer cell lines, especially those that overexpress Mdm2. While ezetimibe does not structurally resemble nutlins, based on molecular docking simulations ezetimibe accurately mimics p53 binding to the Mdm2 hydrophobic cleft. Ezetimibe, however, has structural vulnerabilities that hinder its use as an anti-cancer agent, such as its conversion by metabolic enzymes in the intestines. This results in negligible bioavailability of ezetimibe in the intestines and as such it is unsuitable for use in the treatment of colonic cancers.

In order to address this, the applicant was prompted to modify ezetimibe in order to decrease its degradation by metabolic enzymes in the intestine. This was achieved by replacing a hydroxyl group, which is a site for glucuronidation, in the ezetimibe lead molecule with a fluorine. This new drug, MC011019, has improved drug-like properties. The modification prevents metabolic transformation of the drug in the intestine and increases its bioavailability in the intestine, which makes it suitable for treating colon and colorectal cancers, and potentially other cancers that overexpress Mdm2.

### DISCLOSURE OF THE INVENTION

According to a first aspect of the invention there is provided a compound having the structure of Formula (I):

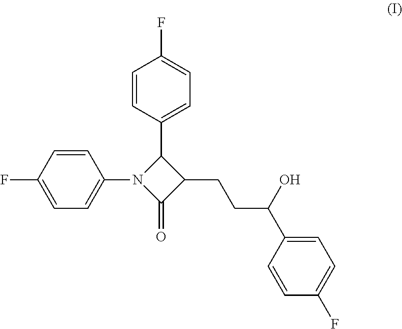 [SMILES: O=C1C(CCC(O)c2ccc(F)cc2)C(c2ccc(F)cc2)N1c1ccc(F)cc1]

or a pharmaceutically acceptable salt thereof.

The compound or a pharmaceutically acceptable salt thereof may bind tightly to Mdm2, and in particular to the hydrophobic binding pocket of Mdm2 to which p53 binds. The compound or a pharmaceutically acceptable salt thereof may therefore prevent the binding of Mdm2 to p53, thereby increasing the levels of p53 in a cell.

The compound or a pharmaceutically acceptable salt thereof may have good bioavailability in the intestine as it may resist degradation, and in particular glucuronidation.

According to a second aspect of the invention there is provided a compound of Formula (I) or a pharmaceutically acceptable salt thereof for use in a method of treating a cancer.

The cancer may be a cancer in which there are elevated levels of Mdm2 or where Mdm2 is overexpressed and may include colon cancer, colorectal cancer, sarcoma, glioma, lymphoma, breast cancer, lung cancer, liver cancer, esophagogastric cancer and gynaecological cancers.

The compound or a pharmaceutically acceptable salt thereof may bind to the hydrophobic binding pocket of Mdm2 and increase the levels of active p53 in a cell. This may increase p53-mediated cell death of cancer cells.

According to a third aspect of the invention there is provided use of a compound of Formula (I) or a pharmaceutically acceptable salt thereof in the manufacture of a medicament for treatment of a cancer, which may be a cancer in which Mdm2 levels are high or it is overexpressed.

The cancer may include colon cancer, colorectal cancer, sarcoma, glioma, lymphoma, breast cancer, lung cancer, liver cancer, esophagogastric cancer and gynaecological cancer.

The compound or a pharmaceutically acceptable salt thereof may bind to the hydrophobic binding pocket of Mdm2 to prevent the inhibition of p53 by Mdm2, thereby increasing p53-mediated cell death of cancer cells.

According to a fourth aspect of the invention there is provided a method of treating a cancer by administering the compound of Formula (I) or a pharmaceutically acceptable salt thereof to a patient in need thereof, wherein the cancer may be characterised by high levels or overexpression of Mdm2.

The compound or a pharmaceutically acceptable salt thereof may bind to the hydrophobic binding pocket of Mdm2 and increase the levels of p53 in the cell. The compound or a pharmaceutically acceptable salt thereof may promote p53-mediated cell death of cancer cells.

The cancer may be selected from colon cancer, colorectal cancer, sarcoma, glioma, lymphoma, breast cancer, lung cancer, liver cancer, esophagogastric cancer and gynaecological cancer.

### EXAMPLE

The invention will now be described in more detail with reference to the Example hereunder, and the accompanying drawings.

In the drawings

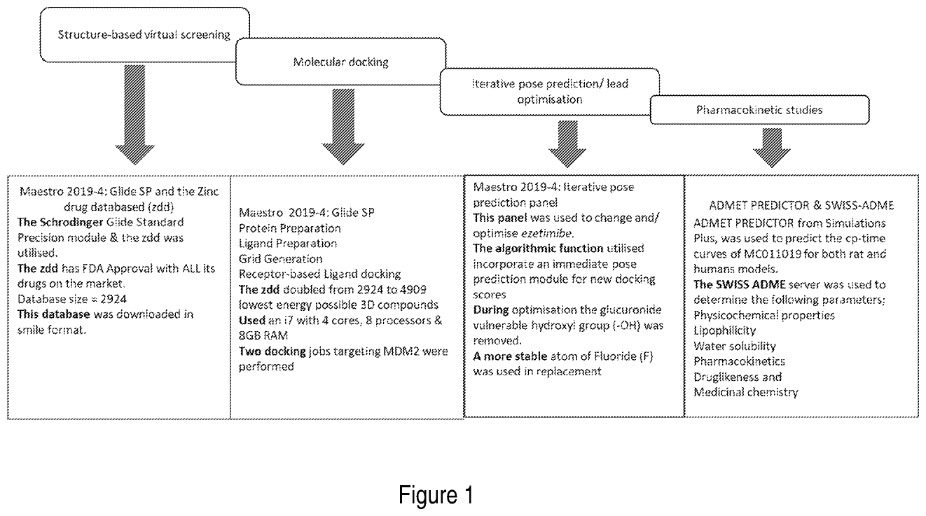

FIG. 1 shows, for the Example, a summary of the adopted structure guided computer aided drug design methodology;

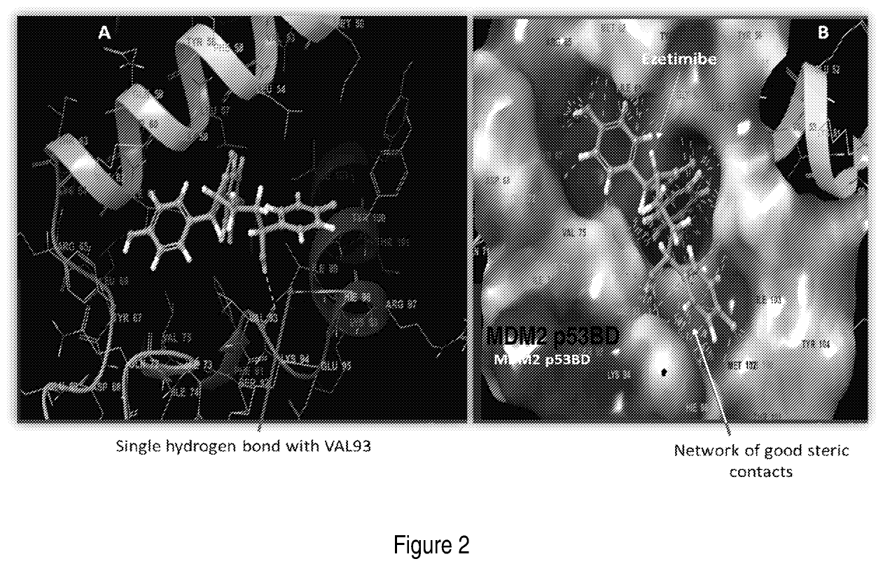

FIG. 2 shows, for the Example, (A) ezetimibe and Mdm2 interaction and (B) a residue property surface along with a network of good steric contacts of the ezetimibe-Mdm2 complex;

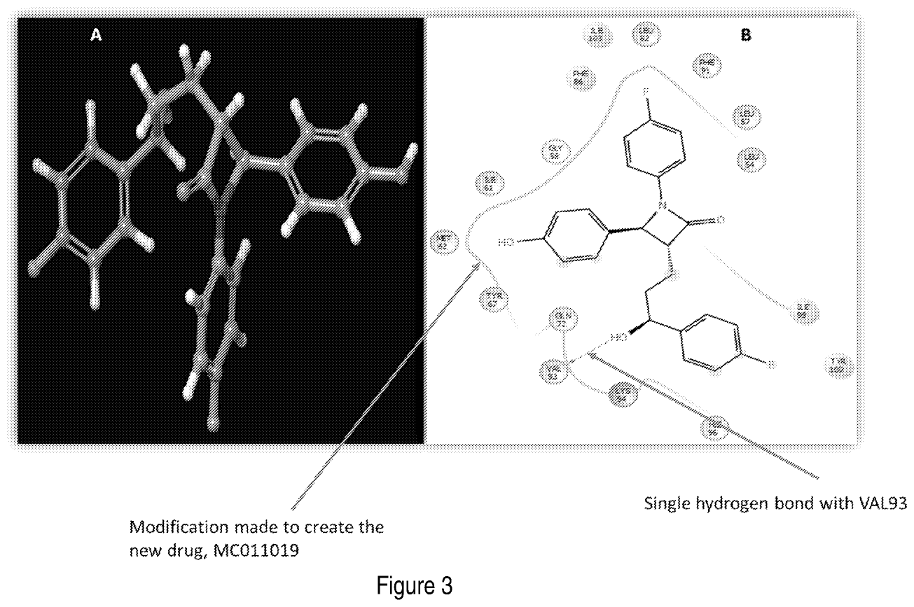

FIG. 3 shows, for the Example, the structural composition and interaction analysis of Mdm2 with ezetimibe with (A) being a ball and stick model of ezetimibe (ZINC03810860) and (B) being a 2D ligand interaction diagram with a collection of nearby residues and a single hydrogen bond co-ordination to VAL93;

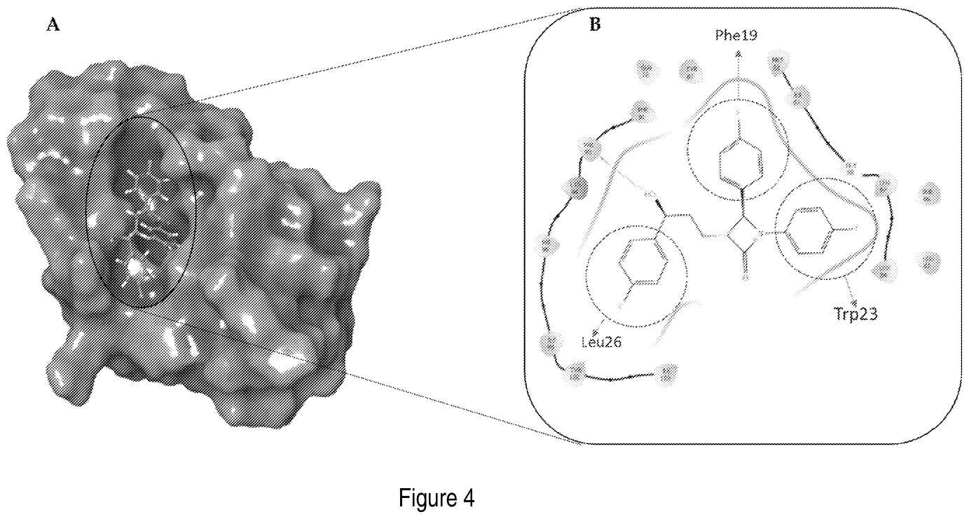

FIG. 4 shows, the simulated surface presentation and interaction analysis of the Mdm2-MC011019 complex, with (A) being the Mdm2-MC011019 complex, where Mdm2 is shown in a grey surface area with a deep hydrophobic pocket and MC011019 is represented in ball and stick models; (B) being a 2D ligand interaction diagram with a collection of nearby residues at 3 Å axis and a single hydrogen bond co-ordination to VAL93; and wherein the para fluoro-phenyl groups of MC011019 that are predicted to competitively inhibit the three critical residues of Phe19, Trp23 and Leu26 of the p53 transactivation domain are circled and labelled respectively;

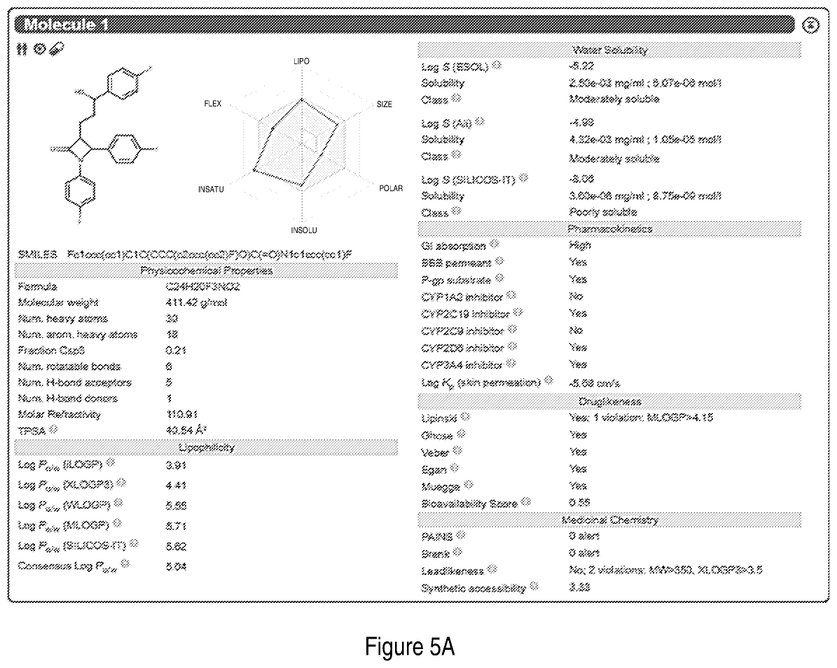

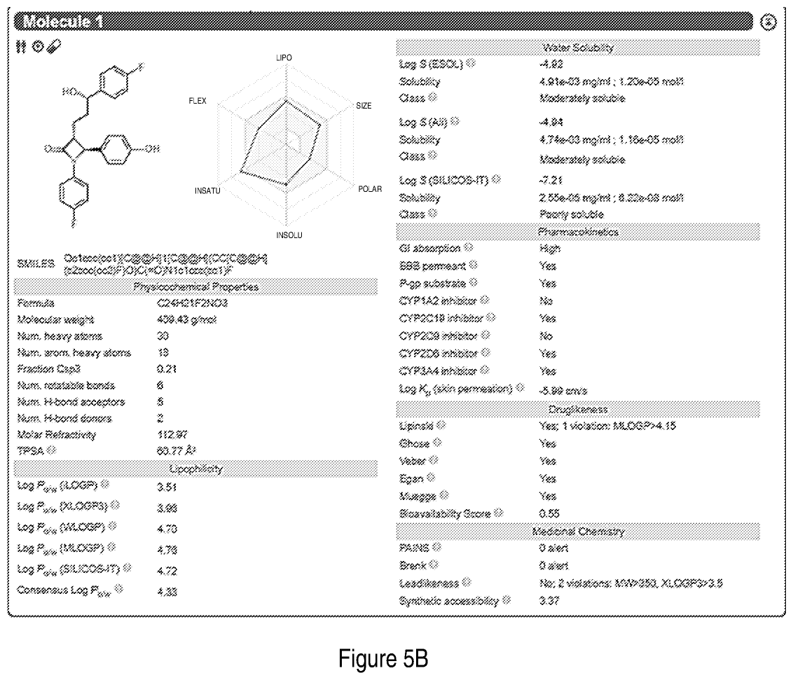

FIG. 5 shows pharmacokinetic (PK) properties of MC011019 (A) compared with ezetimibe (B);

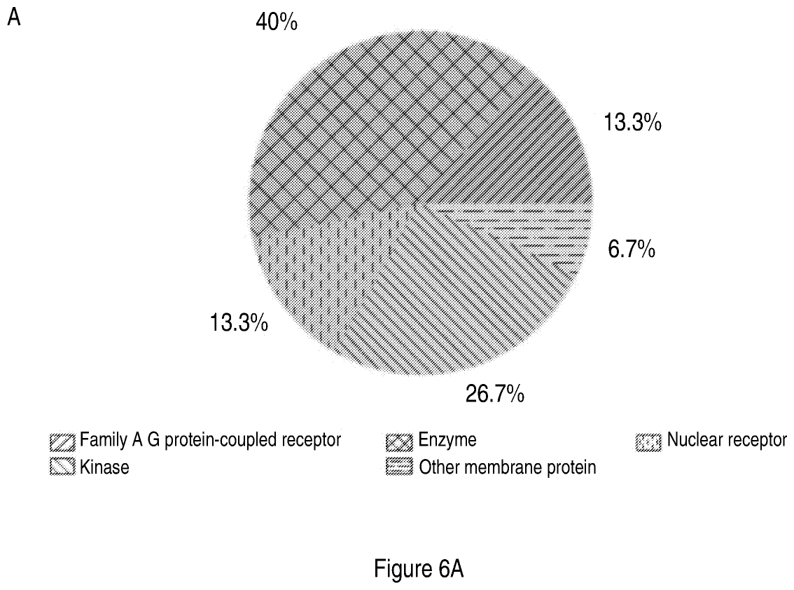

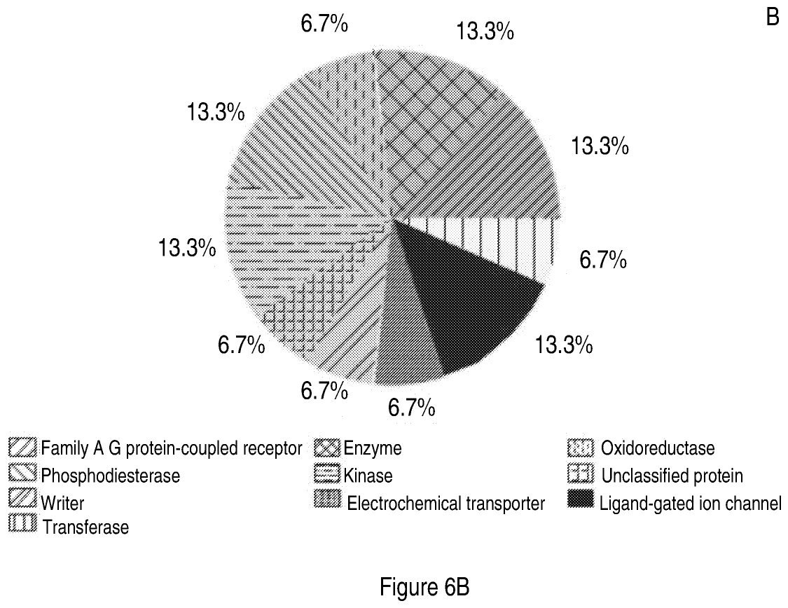

FIG. 6 shows, for the Example, a target prediction analysis with (A) being a summary of ezetimibe protein targets and (B) being a summary of possible MC011019 protein targets;

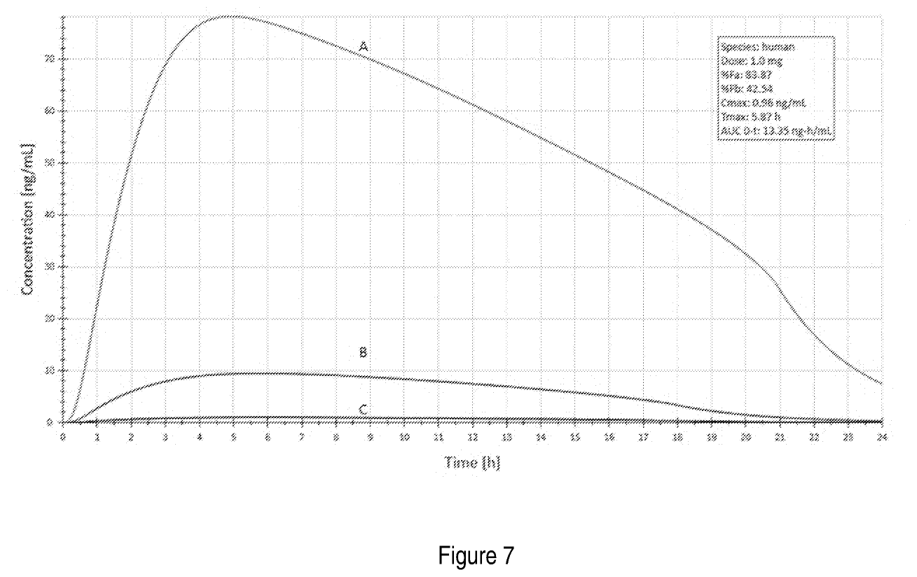

FIG. 7 shows, for the Example, the pharmacokinetic profile in terms of cp-time curve analysis of MC011019 illustrating a prediction of the possible plasma concentration curve (cp-time curve) of MC011019 administered at different doses, i.e. 0.01 mg (C), 0.1 mg (B) and 1.0 mg (A) using the rat as a model, the percentage of fraction absorbed (% Fa) and bioavailable (% Fb) as well as the C-max with its corresponding T-max;

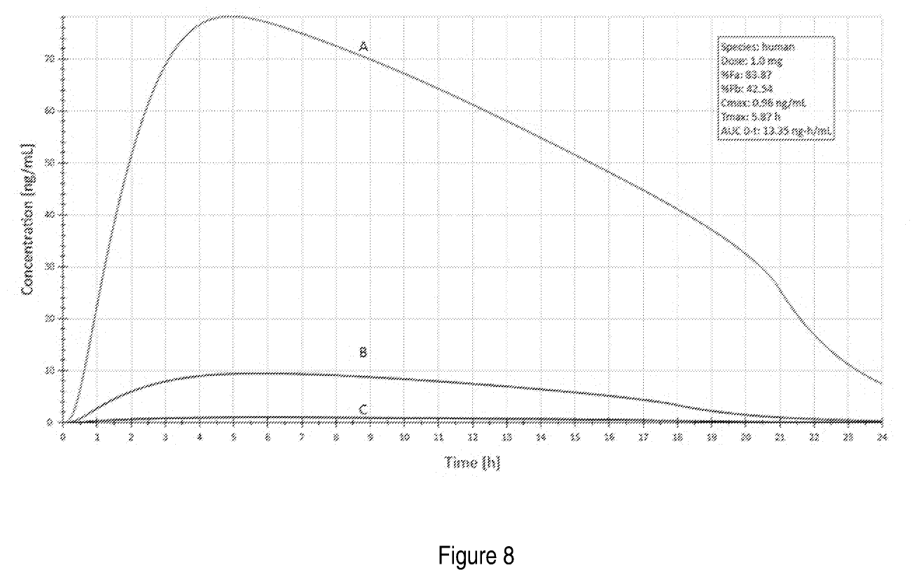

FIG. 8 shows, for the Example, the pharmacokinetic profile in terms of cp-time curve analysis of MC011019, illustrating a prediction of the possible plasma concentration curve (cp-time curve) of MC011019 administered at different doses, i.e. 1 mg (C), 10 mg (B) and 100 mg (A) using a human model, the percentage of fraction absorbed (% Fa) and bioavailable (% Fb) as well as the C-max with its corresponding T-max; and

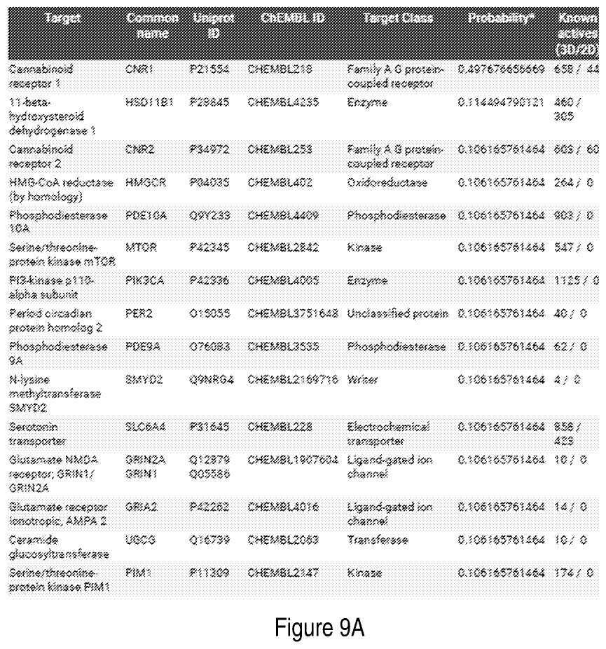

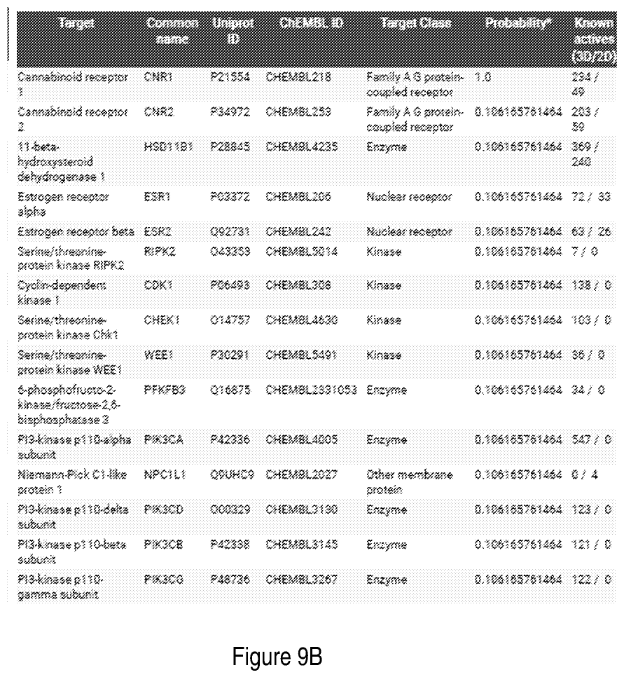

FIG. 9 shows, for the Example, (A) the probability of MC011019 to interact off-target with various cellular proteins, and (B) the probability of ezetimibe to interact off-target with various cellular proteins.

### MATERIALS AND METHODS

Protein and Drug Structures

The Mdm2 protein structure was downloaded from the Protein Database (PDB) in a pdb format and analysed using PyMol on which the p53 peptide was removed prior to docking studies. The drug ligands structures were retrieved from the Zinc Drug Database (Zdd) in a 2D configuration. The PubChem database was utilized to obtain the nutlin-3a drug structure in sdf format. The structure of ezetimibe was obtained from the DrugBank database in a SMILE format.

Screening of Chemical Compounds and Molecular Docking

The Mdm2 p53-binding domain (Mdm2 p53BD) was used as a template on the Schrödinger's Maestro 2019-4: Glide SP (Standard Precision) application to screen the Zdd for chemical compounds that can target the Mdm2 p53BD. The Zdd constitutes commercially FDA approved drugs, available worldwide as pure compounds. In this study, the entire Zdd database of 2924 structures was screened. The use of Glide enabled both virtual screening and molecular docking studies to be done simultaneously. This application took the critical residues within the Mdm2 p53BD and the Zdd as inputs and generated a collection of chemical compounds docked into the specified pocket with different docking scores. The Schrödinger's Receptor-based ligand docking protocol employs a multi-step procedure, which involves the preparation and manipulation of the Mdm2 p53BD as well as ligands prior to screening and docking studies. These steps were sequentially performed as follows: Protein domain preparation, Ligand Preparation, Grid generation and Receptor-based ligand docking.

Protein Preparation

The PDB structures are not suitable for immediate use in molecular modelling calculations, as they usually consist of only heavy atoms. They may also include core crystallized ligand, water molecules, metal ions and co-factors. Additionally, some structures are multi-meric and need to be reduced to a single unit, and because of the limited resolution of X-ray experiments it can be difficult to distinguish between the carbonyl oxygen and the secondary amine nitrogen of the amides in crystal structures thus the placement of the groups must be checked. PDB structures may also be missing atoms or connectivity information which must be assigned along with bond-orders and formal charges. Therefore, in this study the Schrödinger-Maestro v10.7 protein preparation wizard was used. This took Mdm2 p53BD from their raw state (having missing atoms or incorrect bond-order assignments, incorrect charge states and orientation of various groups) and brought them to a suitable state of being utilised by Glide. The wizard contains a Graphical User Interphase (GUI) with a systematic functional procedure.

Ligand Preparation

The Zdd was downloaded (http://zinc12.docking.org/browse/subsets/special) in SMILE and SDF formats which contained 2D structures of chemical compounds. This configuration state is not suitable when performing molecular docking calculations, or to simulate using computational docking algorithms. Proteins exist in 3-dimensional space, thus drugs that would successfully target them should also exhibit such configuration. The Schrödinger-Maestro v10.7 ligand preparation wizard was used to convert 2924 2D structures into lowest energy possible 4909 3D structures in maestro format. This program allows for an expansion of each input structure by generating variations on the ionisation state, tautomers, stereochemistry, and ring conformations. The possible ionisation states of the ligands were generated at a target pH range of 7.0+/−2 using Epik which is a built-in application within Glide that predicts both the ionisation states and their associated penalties. Epik also predicts different tautomeric forms and calculates energy penalties for every ligand state it predicts.

In Glide, the Epik state penalty is also used to differentiate active from inactive compounds during docking, in fact the use of Epik is known to improve virtual screening enrichment.

Grid Generation

The outer scoring grids were generated with different dimensions ranging from 20×20×20 Å to 50×50×50 Å in the x, y, z-axis respectively. Generally, it is important to make the outer grid consistent with the shape of the protein's active site, thus this was done to only cover-up the active site volume of the Mdm2 p53-binding hydrophobic cleft. Literature-stipulated residues of the Mdm2 p53BD were used as a premise to accurately map-out critical residues facilitating the binding co-ordination of the p53 transactivation domain. A ligand centre box (inner grid) was generated to define the acceptable ligand centre positions during the side point search, providing a true measure of the effective search space size. The ligand centre box is useful for ligands to find usual or asymmetric binding modes in the active site or to confine their midpoints into a smaller box to save calculation time. The “centroid of selected residues” option, which specifies the residues that best define the active site was also used, and the inner grid was then centred on the centroid of these selected residues.

Receptor-Based Ligand Docking

The final docking algorithm utilised in this study is the Glide SP-algorithm, better known as the standard precision. The nature of docking simulations employed by this algorithm are the same to that of the High Throughput Virtual Screening (HTVS), except that HTVS reduces the number of intermediate conformations throughout the docking funnel, the thoroughness of the final torsional refinement and sampling. During the docking process, the domain-structures were kept rigid (not even the hydroxyl and thiol groups could rotate), and flexibility was induced to all docking ligands. This was achieved through the ligand preparation wizard, which had generated a collection of multiple poses of the ligand database (Zdd). The entire work was done using a Core i7 with 4 cores, 8 processors, and 8 GB of RAM.

Results

Screening of the Zdd revealed very good binding of ezetimibe to the Mdm2-p53 binding pocket including a hydrogen bond with VAL93. Further molecular docking studies produced the interaction elements shown in FIG. 2 where ezetimibe is docked into the p53-binding domain of Mdm2 i.e. the hydrophobic pocket. In FIG. 2A a single hydrogen bond with VAL93 and possible hydrophobic interactions are observed. In FIG. 2B a residue property surface along with a network of good steric contacts of the Mdm2-ezetimibe complex is observed. This surface representation shows the pocket-like nature of the binding site and how ezetimibe accurately mimics the three critical p53 binding residues (PHE19, TRP23, and LEU26).

The ligand interaction diagram (FIG. 3) shows that ezetimibe binds tightly to the Mdm2-p53BD hydrophobic pocket. Furthermore, it shows ionic interactions at 3A axis around the drug (FIG. 2). This finding is likely to have significant impact in cancers with high Mdm2 expression. A fluorine is introduced as shown by the square in FIG. 3 and in silico pharmacokinetic studies were conducted. This substitution is critical because it blocks the conversion of the new drug by glucuronidation in the intestines. This modification also improves the druglikeness of the lead compound. One piece of evidence is that the lipophilicity (C log P) of the drug is increased to 5.04. For ezetimibe lipophilicity did not improve oral bioavailability because after glucuronidation, ezetimibe is excreted through the digestive tract.

In FIG. 4 the structural interaction of MC011019 docked into the MDM2's hydrophobic pocket is depicted, together with the 2D ligand interaction diagram which shows the binding co-ordination at 3 Å axis. This binding mode accurately mimics the p53 transactivation domain because the three para fluoro-phenyl groups of MC011019 directly bind and occupy the three critical residues (Phe19, Trp23 and Leu26) that facilitate the binding co-ordination of p53. In addition, MC011019 also forms a hydrogen bond with VAL93 similar to that formed by p53 upon binding the MDM2 hydrophobic pocket. Furthermore, this binding mode has a slightly higher docking score of −7.89 kJ/mol compared to the −7.76 kJ/mol depicted by ezetimibe.

In FIG. 5 the pharmacokinetics (PK) and drug-likeness of MC011019 (FIG. 5A) and ezetimibe (FIG. 5B) are illustrated. The figure was computed using Swiss-ADM E.

A target prediction experiment shows that MC011019 and ezetimibe have similar biological targets (FIG. 6, FIG. 9A and FIG. 9B). It is noteworthy that MC011019 is not predicted to bind to the ezetimibe cholesterol-related receptor Niemann-Pick C1 Like protein. This means that MC011019 will probably not replicate the current application of ezetimibe.

Alternatively, cholesterolaemia is unlikely to be an indication for MC011019 as this depends upon binding to the Niemann-Pick C1 Like protein. Furthermore, the probability of MC011019 to bind cannabidiol receptor 1 has also decreased quite significantly when compared to that of ezetimibe.

In FIGS. 7 and 8 the plasma concentration vs time graphs of MC011019 is depicted. FIGS. 7 and 8 were computed using ADM ET PREDICTOR v9.5 from Simulation Plus, Inc. These graphs illustrate the pharmacokinetic profile prediction of MC011019 administered at different doses, i.e. 0.01 mg or 1 mg (C), 0.1 mg or 10 mg (B) and 1.0 mg or 100 mg (A) using the rat and human models respectively. This simulation also computes the percentage of fraction absorbed (% Fa) and bioavailable (% Fb) as well as the C-max with its corresponding T-max. It is also worth noting that in both models MC011019 has shown a great absorption and elimination rates, as well as a good % Fb in humans. These observations are also supported by the findings in FIG. 6, FIG. 9A and FIG. 9B which show that MC011019 does not bind the Niemann-Pick C1 Like 1 protein and further possess a very low probability to interact off-target with other cellular proteins.

Discussion

The in-silico studies show that MC011019 binds strongly in the Mdm2-p53 hydrophobic pocket. The increased lipophilicity (Log P_{o/w}) indicates that MC011019 will have a better bioavailability than the parent molecule ezetimibe due to the addition of the fluorine. The replacement of the hydroxyl by the fluorine is necessary to prevent the metabolic conversion of the drug in the small intestine. Another significant observation is that MC011019 probably does not interact with the Niemann-Pick C1 protein which facilitates absorption of cholesterol.

These studies suggest that MC011019 will prevent the binding of Mdm2 to the tumour suppressor protein p53 thereby reactivating p53 for its positive action on cancer cells. It is anticipated that MC011019 will be effective against wild type p53 cancers and in cancers that overexpress Mdm2. In particular, it is anticipated that MC011019 will be effective in targeting colon and colorectal cancers since it is not vulnerable to degradation by intestinal metabolism.

1. A compound having the structure of Formula (I): 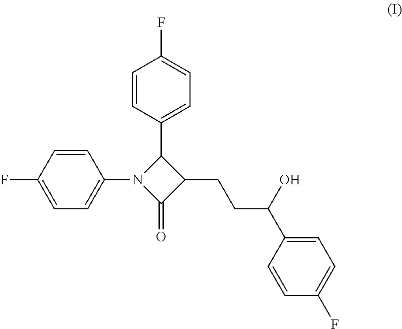 [SMILES: O=C1C(CCC(O)c2ccc(F)cc2)C(c2ccc(F)cc2)N1c1ccc(F)cc1]
or a pharmaceutically acceptable salt thereof.

2. The compound of claim 1, or a pharmaceutically acceptable salt thereof, wherein the compound binds to a p53 binding pocket of Mdm2 to prevent binding of Mdm2 to p53 and increase p53 levels in a cell.

3. A compound of Formula (I), or a pharmaceutically acceptable salt thereof, for use in a method of treating a cancer in which there are elevated levels of Mdm2 or where Mdm2 is overexpressed.

4. The compound, or a pharmaceutically acceptable salt thereof, for use of claim 3, wherein the cancer is selected from colon cancer, colorectal cancer, sarcoma, glioma, lymphoma, breast cancer, lung cancer, liver cancer, esophagogastric cancer and gynaecological cancer.

5. The compound, or a pharmaceutically acceptable salt thereof, for use of claim 3, wherein the compound or a pharmaceutically acceptable salt thereof binds to a p53 binding pocket of Mdm2 to increase levels of p53 in a cell.

6. Use of the compound of Formula or a pharmaceutically acceptable salt thereof, in the manufacture of a medicament for treatment of an Mdm2 cancer, wherein the cancer is selected from colon cancer, colorectal cancer, sarcoma, glioma, lymphoma, breast cancer, lung cancer, liver cancer, esophagogastric cancer and gynaecological cancer, and wherein the compound binds to a p53 binding pocket of Mdm2 to increase levels of p53 in a cell.
